(19) United States
(12) Patent Application Publication
(10) Pub. No.: US 2013/0006121 A1
(43) Pub. Date: Jan. 3, 2013

(54) Device for Identifying Hemodynamic Changes

(75) Inventor: Stuart Hopper Myers, Baltimore, MD (US)

(73) Assignee: THE JOHNS HOPKINS UNIVERSITY, Baltimore, MD (US)

(21) Appl. No.: 13/634,330

(22) PCT Filed: Mar. 16, 2011
(86) PCT No.: PCT/US11/28708

§ 371 Date: Sep. 12, 2012

Related U.S. Application Data
(60) Provisional application No. 61/314,242, filed on Mar. 16, 2010.

(51) Int. Cl. A61B 5/0295; A61B 6/00

(52) U.S. Cl. 600/479; 600/507

(57) ABSTRACT

A device for identifying changes in or elevated levels of compartment pressure has a blood-volume sensor adapted to be arranged at a distal blood-flow region relative to a region of interest, and a data processor in communication with the blood-volume sensor. The data processor is configured to receive a data signal from the blood-volume sensor, obtain a reference signal, compare the data signal to the reference signal to identify a difference between the reference signal and the data signal that is indicative of an change in pressure within the region of interest, and identify at least one of changes in or elevated levels of compartment pressure in the region of interest based on the comparing.

### CROSS-REFERENCE OF RELATED APPLICATION

This application claims priority to U.S. Provisional Application No. 61/314,242, filed Mar. 16, 2010, the entire contents of which are hereby incorporated by reference.

### BACKGROUND 1. Field of Invention

The field of the currently claimed embodiments of this invention relates to systems, methods and software for monitoring and detecting elevated compartment pressure, and more particularly non-invasive systems and methods for monitoring and detecting elevated compartment pressure.

2. Discussion of Related Art

Compartment syndrome is the compression of nerves, blood vessels, and muscle inside a closed space within the body. The compression of blood vessels by the raised pressure within the compartment can lead to tissue death from a lack of oxygen. Compartment syndrome most often involves the forearm and lower leg and is most often caused by trauma, especially that involving fractures and/or crushing types of injuries. In extreme cases, compartment syndrome can lead to the loss of limbs or even death.

The current method for the diagnosis of compartment syndrome involves pressure transduction via introduction of a large-bore needle into the fascial compartment of interest. This is poorly tolerated by patients, notoriously unreliable, and can not be practically used for continuous monitoring. There is currently no method for the non-invasive and continuous monitoring of patients at risk for the development of compartment syndrome. Many modalities have been investigated including pulse oximeter derived hemoglobin saturation, near-infrared spectroscopy, and devices to determine compartment hardness. The lack of a non-invasive monitoring device is especially problematic when patients are identified as having injuries which place them at high risk for the development of compartment syndrome (e.g., tibia fractures, crush injuries, etc.). Therefore, there remains a need for improved devices and methods to monitor for compartment syndrome.

### SUMMARY

A device for identifying changes in or elevated levels of compartment pressure according to an embodiment of the current invention has a blood-volume sensor adapted to be arranged at a distal blood-flow region relative to a region of interest, and a data processor in communication with the blood-volume sensor. The data processor is configured to receive a data signal from the blood-volume sensor, obtain a reference signal, compare the data signal to the reference signal to identify a difference between the reference signal and the data signal that is indicative of an change in pressure within the region of interest, and identify at least one of changes in or elevated levels of compartment pressure in the region of interest based on the comparing.

A method of detecting and monitoring changes in or elevated levels of compartment pressure according to an embodiment of the current invention includes arranging a blood-volume sensor at a distal blood-flow region relative to a region of interest, the blood-volume sensor providing a data signal; obtaining a reference signal; comparing the data signal to the reference signal to identify a difference between the reference signal and the data signal that is indicative of a change in pressure within the region of interest; and identifying at least one of changes in or elevated levels of compartment pressure in the region of interest based on the comparing.

A computer-readable medium according to an embodiment of the current invention includes software that when executed by a computer causes the computer to perform processes including receiving a data signal from a blood-volume sensor arranged at a distal blood-flow region relative to a region of interest; obtaining a reference signal; comparing the data signal to the reference signal to identify a difference between the reference signal and the data signal that is indicative of a change in pressure within the region of interest; and identify at least one of changes in or elevated levels of compartment pressure in the region of interest based on the comparing.

A device for identifying hemodynamic changes according to an embodiment of the current invention has a blood-volume sensor adapted to be arranged at a distal blood-flow region relative to a region of interest, and a data processor in communication with the blood-volume sensor. The data processor is configured to receive a data signal from the blood-volume sensor, obtain a reference signal, compare the data signal to the reference signal to identify a difference between the reference signal and the data signal that is indicative of an hemodynamic change within the region of interest, and identify the hemodynamic change in the region of interest based on the comparing.

### BRIEF DESCRIPTION OF THE DRAWINGS

Further objectives and advantages will become apparent from a consideration of the description, drawings, and examples.

FIG. 1 is a schematic illustration of a device for identifying changes in or elevated levels of compartment pressure according to an embodiment of the current invention.

FIG. 1A is a schematic illustration of a device for identifying changes in or elevated levels of compartment pressure according to another embodiment of the current invention.

FIG. 1B is a schematic illustration of a device for identifying changes in or elevated levels of compartment pressure according to another embodiment of the current invention.

FIG. 1C is a schematic illustration of a device for identifying changes in or elevated levels of compartment pressure according to another embodiment of the current invention.

FIG. 1D is a schematic illustration of a device for identifying changes in or elevated levels of compartment pressure according to another embodiment of the current invention.

FIG. 2 shows an example of the waveforms we captured according to an embodiment of the current invention. These are the six waveforms from one subject. This figure illustrates the change in waveform that occurs as a function of cuff pressure.

FIG. 3 shows the amplitude of the waveform as a function of pressure. Clearly, the amplitude decreases as pressure increases. The rightmost data point in each series is the contralateral “no cuff” amplitude.

FIG. 4 shows a graphical representation of data as it is recorded from the NOVAMETRIX OXYPLETH 520A pulse oximeter.

FIG. 5 shows a graphical representation of the captured data after amplitude normalization based on average deviation from the mean.

FIG. 6 shows spliced waves laid on top of one another.

FIG. 7 shows a characteristic wave (+/−1 SD) from one subject at one specific pressure.

FIG. 8 shows characteristic waves from subject 1. Clearly, there is a change in waveform shape as pressure increases.

FIG. 9 shows characteristic waves at a given pressure from all 10 subjects which have been averaged to create characteristic waves at each pressure for the entire cohort. There is an effect of pressure on the waveform amongst all subjects.

FIG. 10 shows the same information as FIG. 9, but was created using type 1 normalization.

FIG. 11 shows the composite characteristic curves (data compiled from all 10 subjects) at each pressure minus the “R No cuff” composite curve. This illustrates the utility of using the contralateral extremity as a control according to an embodiment of the current invention. Curve subtraction requires normalization of the curves.

FIG. 12 shows the composite normalized curve slope at each pressure level. One of the clearest relationships revealed here is the effect of pressure on the max downslope at the beginning of the curve. The left and right “No Cuff” signals also tend to have similar peak downslopes.

FIG. 13 shows composite curve y-intercept (type 2 normalization) as a function of pressure.

FIG. 14 shows a more detailed examination of the slope of the normalized waves around the area where their slope is the most negative. As pressure increases, this slope decreases.

FIG. 15 shows an exponential regression to the first three data points of the normalized characteristic curves at each pressure level for each subject as a function of pressure.

FIG. 16 shows one of the curve shape features used in the multiple regression analysis. The chart shows the relationship between the Δy from x=0 to x=17 on the normalized characteristic curves at each pressure level for each subject.

FIG. 17 shows the results of a multiple linear regression analysis with a backward stepwise independent variable entry. The variables consisted of approximately 50 normalized curve features. The x axis is the cuff pressure. The y axis is the predicted pressure based on the regression. The R “No cuff” feature value is subtracted from the experimental pressure feature value prior to being input into the regression model. The regression was able to reliably predict the experimental pressure.

FIG. 18 shows the ROC curve based on a neural network classifier that would predict the pressure for a given waveform.

FIG. 19 shows histograms based on the neural network classifier output for each subject.

FIG. 20 shows a schematic for our pig model data collection.

FIG. 21 shows the relationship between upstroke slope (normalized curves) of the control extremity as a function of experimental extremity compartment pressure in our pig model.

FIG. 22 shows experimental extremity upstroke slope (normalized curves) to the control extremity upstroke slope (normalized curves) as a function of experimental extremity pressure.

FIG. 23 shows the ratio of the experimental extremity upstroke slope (normalized curves) to the control extremity upstroke slope (normalized curves) as a function of experimental extremity pressure.

FIG. 24 shows results of a leave-one-out cross validation of the multiple regression analysis corresponding to FIG. 17.

FIG. 25 shows examples of various waveform parameters.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some embodiments of the current invention can identify and monitor raised intra-compartmental pressure on a continuous and non-invasive basis. According to some embodiments of the current invention, a pulse oximeter plethysmographic waveform is used to identify elevated intra-compartmental pressure. The waveform transduced distal to the affected compartment is attenuated when compared to the contralateral (non-affected) extremity, for example. This difference is the result of compression of vascular structures travelling through the affected compartments. The altered waveform morphology can be quantified in several ways, most simply as a ratio of amplitudes between affected and unaffected extremities. Conventional use of pulse oximeters to diagnose elevated compartment pressure has been limited to the use of calculated oxygen saturation in the affected extremity. However, according to some embodiments of the current invention, pulse oximeter plethysmography is used to monitor compartment pressure.

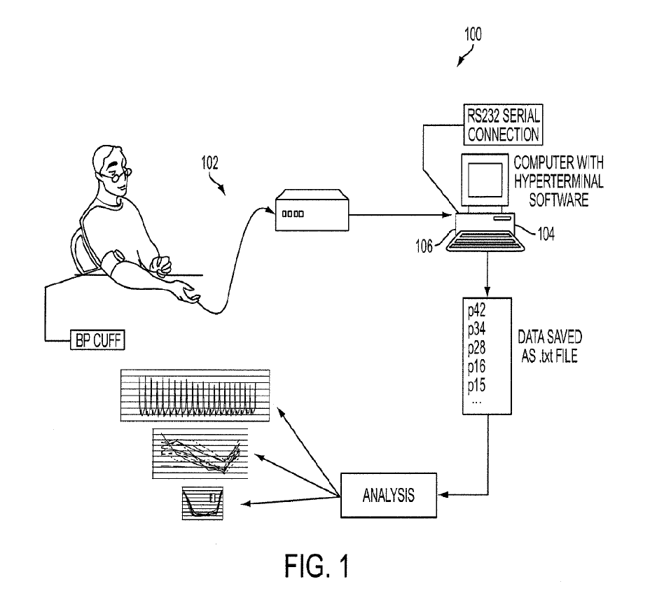

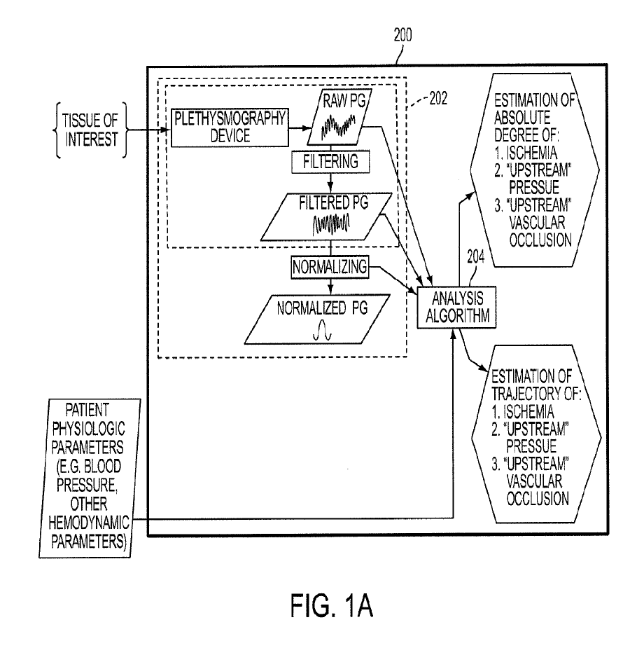

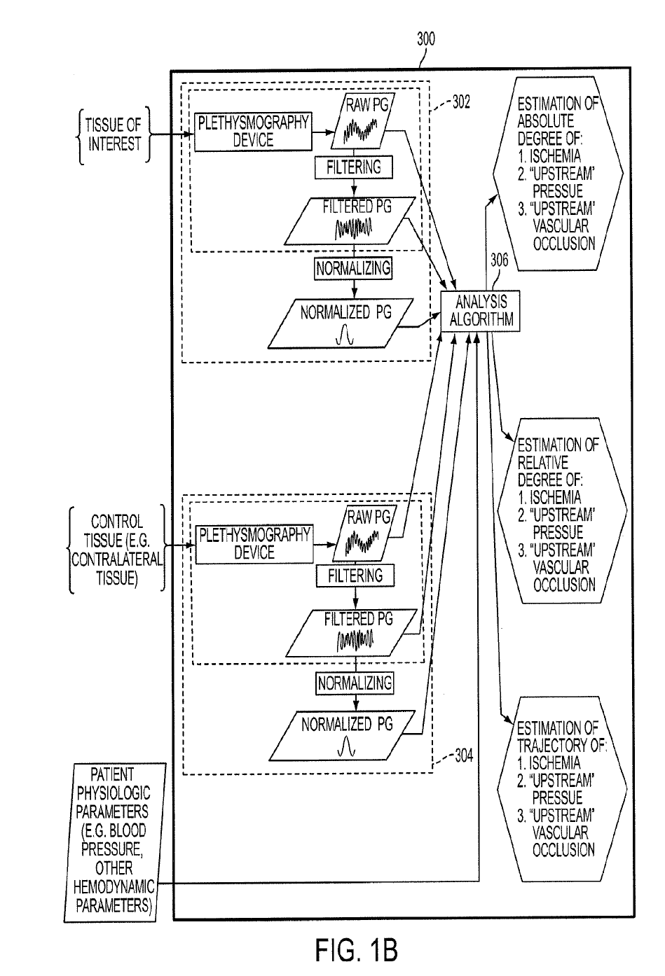

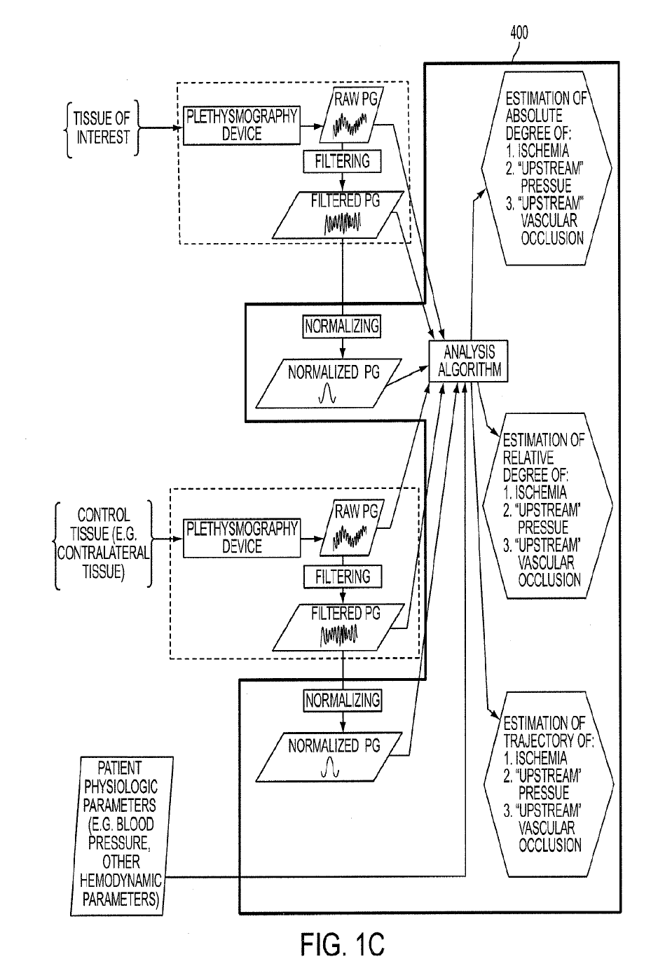

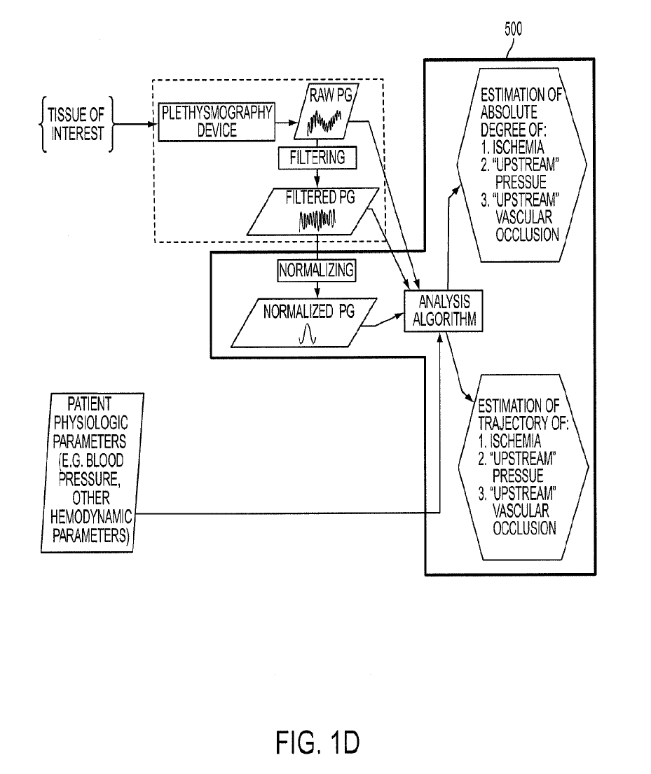

FIG. 1 provides a schematic illustration of a device for identifying changes in or elevated levels of compartment pressure 100 according to an embodiment of the current invention. The device 100 has a blood-volume sensor 102 adapted to be arranged at a distal blood-flow region relative to a region of interest and a data processor 104 in communication with the blood-volume sensor 102. The data processor can be a computer, such as a personal computer, lap top computer or a tablet computer, for example. In other embodiments, the data processor can be specifically designed for the compartment pressure monitoring and detection system 100.

The data processor 104 is configured to receive a data signal from said blood-volume sensor 102, obtain a reference signal, and compare the data signal to the reference signal to identify a difference between the reference signal and the data signal that is indicative of an increase in pressure within the region of interest. The data processor is configured to identify at least one of changes in or elevated levels of compartment pressure in the region of interest based on the comparing.

The device 100 can also include a data storage unit 106 configured to store the reference signal. The data storage unit can be a hard drive or removable data storage according to some embodiments of the current invention. The data storage can also be separate from the data processor, such as over a network or over the internet, for example. The processor 104 is configured to obtain the reference signal from the data storage unit 106. The stored reference signal can include previously obtained baseline data. For example, it could be data previously taken from the same patient either a short time prior to, or long before the monitoring and detection. It could also contain data compiled from many patients and averaged or otherwise processed to provide a suitable reference. In other embodiments, the data processor can be configured to generate the reference signal based on a model. The model could be an empirical or semi-empirical model, for example.

In another embodiment of the current invention, the device 100 also includes a second blood-volume sensor adapted to be arranged at a reference region to provide said reference signal (not shown in the figures). For example, if the region of interest is a person's right leg, one blood-volume sensor could be placed on one of the person's toes on the right foot, while the second blood volume sensor could be placed on one of the person's toes on his left foot. If the region of interest is an arm, the blood-volume sensors can be similarly placed on the person's fingers. The general concepts of the current invention are not limited to these particular examples.

The blood-volume sensor 102 can be a non-invasive blood-volume sensor according to some embodiments of the current invention. For example, the blood-volume sensor can be an optical blood-volume sensor according to some embodiments of the current invention. For example, a photoplethysmography (PPG) can be used in which one or more optical transmitters, such as, but not limited to LEDs and/or lasers provide light that is detected by an optical detector. Optical wavelengths in the red and near infrared regions of the spectrum have been found to be suitable for the blood-volume sensors. The light can be either reflected or transmitted light depending on whether the sensor is operating in transmission or reflection mode. When the blood-volume sensors are placed on fingers and toes, for example, transmission mode detectors have been found to work well. Reflectance-type detectors can be useful where tissue shape makes transmission difficult (skin, for example). The output PPG waveform has both a DC and AC component. The DC component corresponds to an average blood volume, while the AC component corresponds to time-varying effects. A high frequency component correlates with the heart beat, while lower frequency components correlate with breathing and other factors. In the case of the onset of elevated compartment pressure, changes in both the DC and AC components can occur due to the restricted flow of blood into the distal region. Of course, the change in the DC component can be viewed as a longer period AC variation. The term “blood-volume sensor” is intended to be a broad term such that it can include DC and/or AC components of the signal that is affected by the average amount as well as time varying amount of blood in the detection volume. A NOVAMETRIX OXYPLETH 520A system has been found to be suitable for the blood-volume sensors according to an embodiment of the current invention.

In an embodiment of the current invention, the data processor 104 is configured to calculate a first ratio of amplitudes of the data signal and the reference signal at a first time and calculate a second ratio of amplitudes of the data signal and the reference signal at a second time to determine a change of the second ratio relative to the first ratio. Thus, according to one embodiment of the current invention, the amplitude of the data signal waveform is compared to the amplitude of the reference signal waveform. However, other parameters of the data and reference waveforms, other than amplitude, can be compared.

In operation of compartment pressure monitoring and detection system 100, blood-volume sensor 102 is arranged at a distal blood-flow region relative to a region of interest. For example, a non-invasive PPG device can be placed on a finger if the region of interest is in the arm or on a toe if the region of interest is in the leg. In one embodiment, a second PPG system is placed on a reference location, such as a finger or toe on the side that is unaffected by the suspected elevated compartment pressure. In this example, the waveforms from the two PPGs are analyzed to determine changes in blood volume and/or flow at the position distal from the region of interest. In one example, the ratio of the amplitudes of the waveforms from the two PPGs can be calculated. However, the waveforms can be compared based on other parameters, such as parameters related to the waveform shapes, for example.

FIGS. 1A-1D are schematic illustrations of additional embodiments of devices for identifying changes in or elevated levels of compartment pressure according to the current invention. FIG. 1A is a schematic illustration of device 200 for identifying changes in or elevated levels of compartment pressure according to an embodiment of the current invention. The device 200 includes a plethysmograph 202, which can have a transducer that is of the transmission type (e.g. for finger, toe, etc) or reflectance-type (e.g. for skin, mucosa, etc). The transducer can be single or multiple wavelength emission/detection. Various light sources, such as lasers, LEDs, etc. are suitable for particular applications. The plethysmograph 202 can be of the pulse oximeter-type (to isolate arterial blood volume) or of near-infrared spectroscopy-type (to identify volume of all blood), depending on the particular application. In lieu of plethymographic waveforms (volume as a function of time), waveforms that represent blood velocity over time could be captured and analyzed. Laser doppler flowmetry is a modality that could also be used in this capacity. In addition, the plethysmograph 202 can include filtering circuits to filter out particular frequency ranges of the signal waveform as well as normalization of the signal waveform. Such circuitry can be integrated with the plethysmograph 202 or added to it.

The tissue of interest could be any tissue at risk for ischemia/hyperemia, such as a revascularized finger, skin overlying free tissue transfer, gastrointestinal (GI) mucosa; any tissue distal to tissue at risk for increased pressure, such as a toe (e.g. in the case of increased leg pressure), a finger (e.g. in case of elevated forearm pressure); or any tissue supplied by compromised/occluded vasculature, such as a toe/foot (e.g. peripheral vascular disease) or toe/foot (e.g. following peripheral bypass surgery). However, the broad concepts of the current invention are not limited to these particular examples.

The device 200 has a data processor that is configured to perform the analysis algorithm 204. In one embodiment, we use a neural network that has the filtered PG as its inputs. Other algorithms can use the raw PG, the filtered PG, the normalized PG, or a combination thereof to identify characteristics that are indicative of altered blood flow. Additional physiologic data from the patient, such as blood pressure, temperature, etc can also be included in the analysis according to some embodiments of the current invention.

FIG. 1B is a schematic illustration of device 300 for identifying changes in or elevated levels of compartment pressure according to an embodiment of the current invention. The device 300 includes a first plethysmograph 302 and a second plethysmograph 304 incorporated in the device 300 as well as a data processor that is configured to perform an analysis algorithm.

FIG. 1C is a schematic illustration of device 400 for identifying changes in or elevated levels of compartment pressure according to an embodiment of the current invention. The device 400 is adapted to interface with two plethysmograph devices rather than being integrated with the device. This can be useful for interfacing with conventional plethysmograph devices, for example.

FIG. 1D is a schematic illustration of device 500 for identifying changes in or elevated levels of compartment pressure according to an embodiment of the current invention. The device 500 is adapted to interface with a plethysmograph device rather than being integrated with the device, similar to the embodiment of FIG. 1C.

### EXAMPLES

The following examples are applications of some specific embodiments of the current invention. These are not intended to limit the general scope of the invention, which is defined by the claims.

We have collected data under a variety of circumstances, both in humans and in pigs. The first human data was collected using a blood pressure cuff to transiently increase compartment pressure. The experimental setup is illustrated in FIG. 1. The data was captured using the RS232 port on the OXYPLETH 520A pulse oximeter.

The data collection sequence went as follows:
1. We measured the patient's blood pressure
2. Recorded PPG waveform from the left arm with no cuff.
3. Recorded PPG waveform from the left arm with a cuff at 20 mm Hg.
4. Recorded PPG waveform from the left arm with a cuff at 40 mm Hg.
5. Recorded PPG waveform from the left arm with a cuff at 60 mm Hg.
6. Recorded PPG waveform from the left arm with a cuff at 80 mm Hg.
7. Recorded PPG waveform from the right arm with no cuff.

We were able to recruit 10 volunteers for the study. We chose to use the oxypleth 520A for two reasons. Many photoplethysmography devices use an autogain function to normalize the waveform amplitude. The Oxypleth 520A allows the user to disable this function.

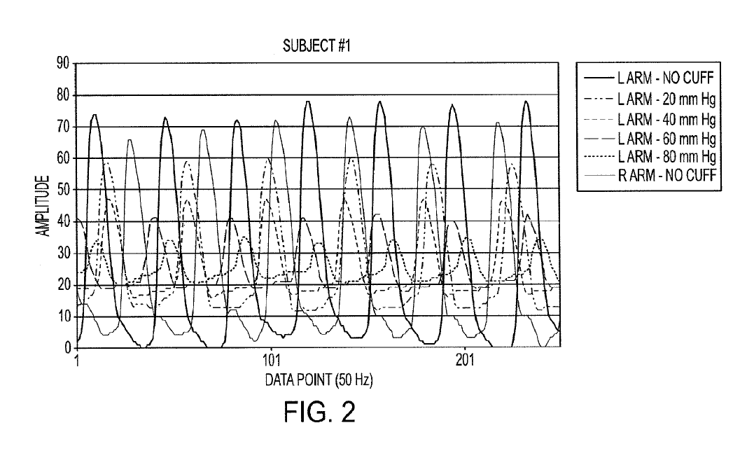

FIG. 2 is an example of the waveforms we captured. These are the six waveforms from subject #1. I have overlaid them to illustrate the change in waveform that occurs as a function of cuff pressure. Notice that the left and right arms are different from each other, despite both having been captured with no cuff in place. It was difficult for us to tell if this was due to inherent asymmetry or if it was an artifact of serial capture. In our second human data collection, we captured signals simultaneously from the left and the right in order to evaluate this apparent asymmetry.

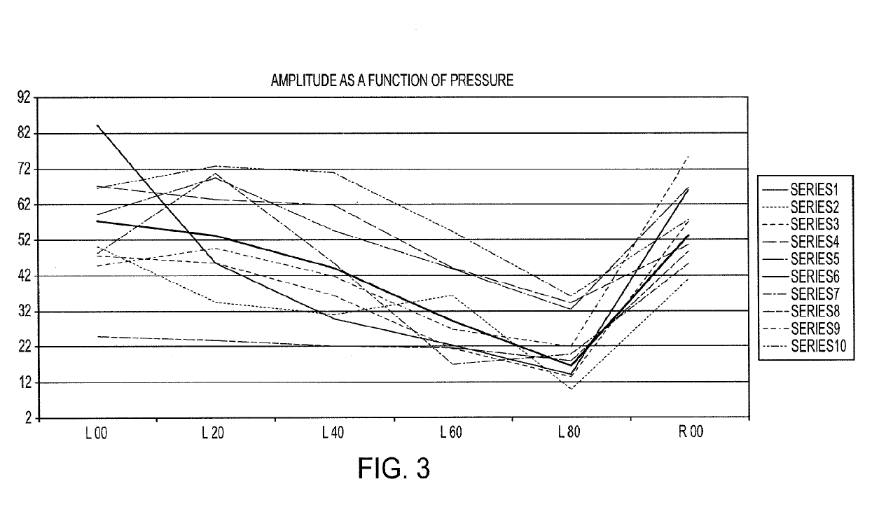

FIG. 3 is a graph of the amplitude of the waveform as a function of pressure. Clearly, the amplitude decreases as pressure increases. The rightmost data point in each series is the contra-lateral “no cuff” amplitude.

To further analyze the data, we wanted to examine the normalized waveforms. Setting the amplitude and period of all the waves to a constant allows comparison of the waveform “shape” characteristic of each subject at each pressure level. This type of analysis is common in the PPG literature.

We used two normalization protocols.

### First Protocol:

1. spliced the data composed of multiple waves into individual waves
2. scaled each waveform so that the amplitude equaled 1. We found the wave peak and trough, then set their values to 1 and 0 respectively.
3. split waveform into 100 data points by interpolation
4. calculated average curves (+/−1 SD) for each subject at each pressure level

### Second Protocol:

1. scaled the series of waveform by dividing each value by the local average deviation from the mean
2. spliced the data composed of multiple waves into individual waves
3. split waveform into 100 data points by interpolation
4. calculated average curves (+/−1 SD) for each subject at each pressure level
   There are many more ways to normalize the waveforms.

We compared the curves qualitatively (looking at them) and quantitatively. Some of the parameters we looked at were curve width, upstroke slope, downstroke slope, area under the curve, upstroke duration, and downstroke duration. I calculated these at 30% max amplitude, 50% max amplitude, and 80% max amplitude. We also calculated linear, exponential, and polynomial regressions for portions of the curves in order to compare curves between subjects and at different pressures.

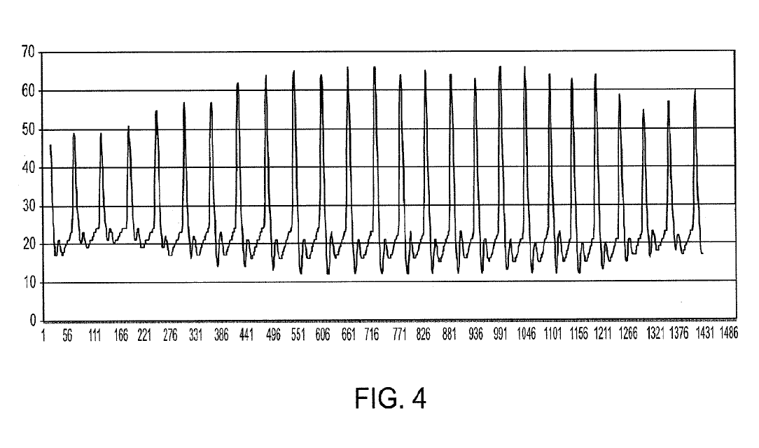

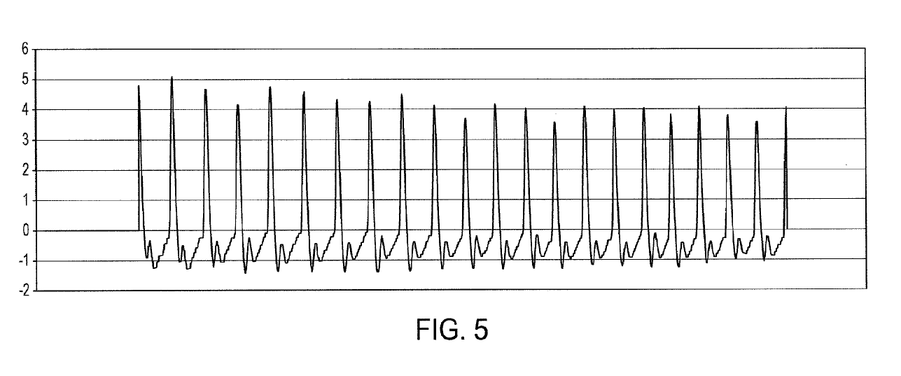

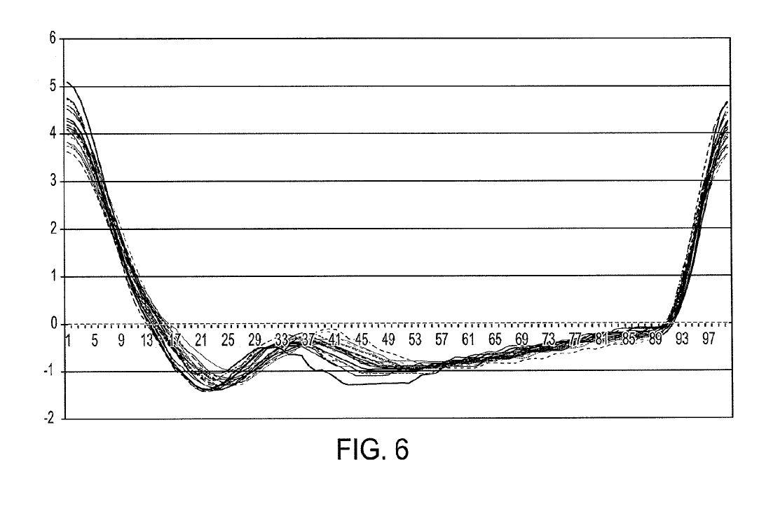

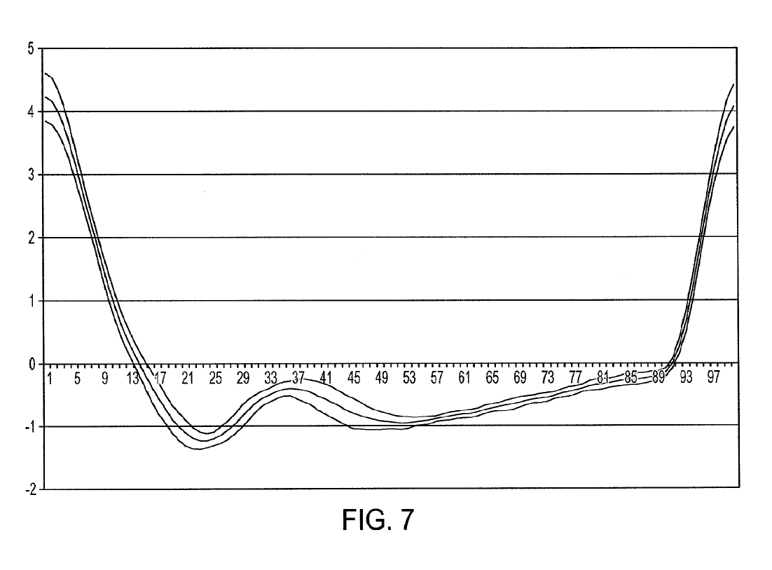

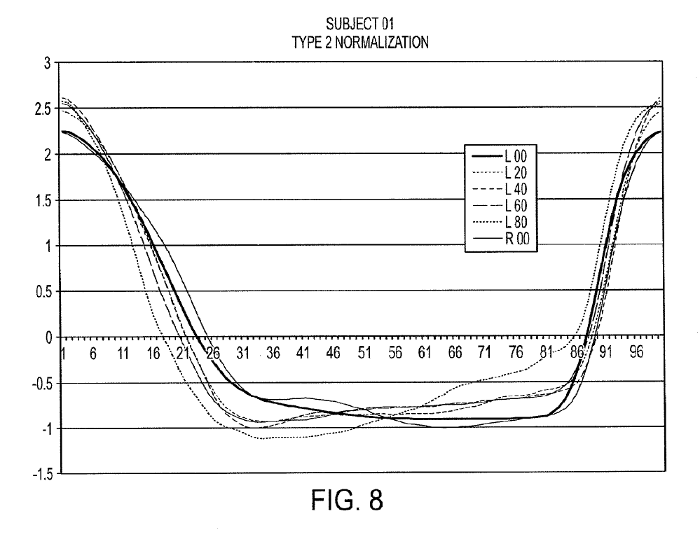

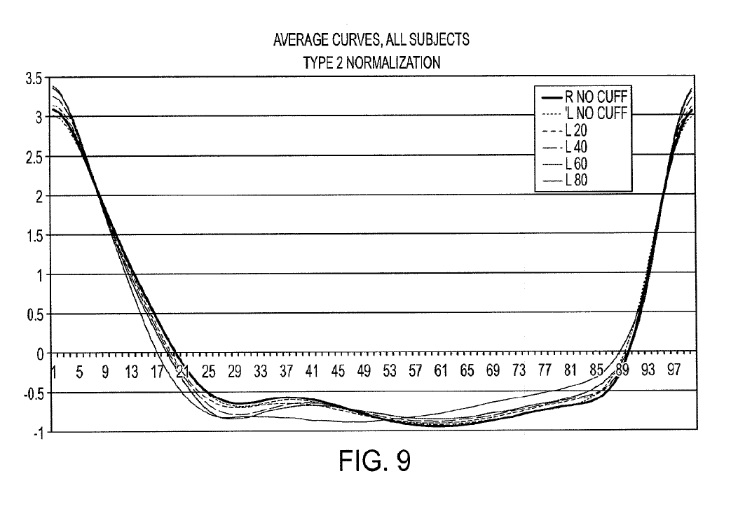

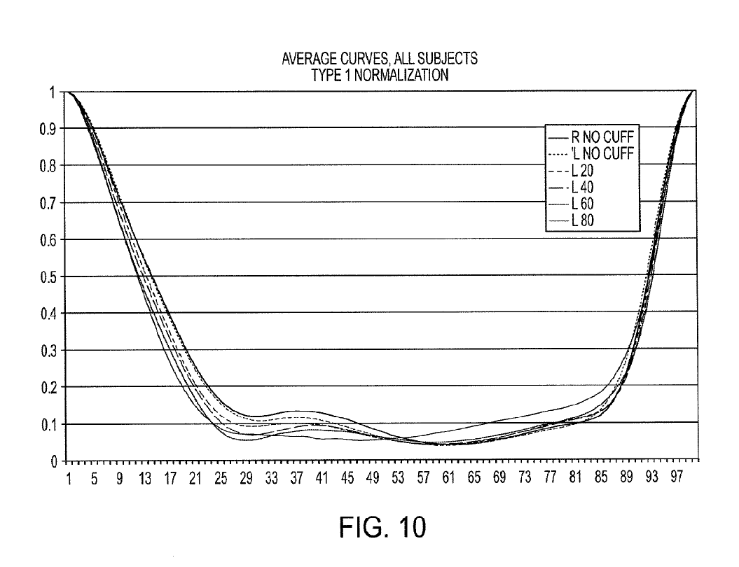

Here are some representative waves from the normalization process (second type of normalization). FIG. 4, shows data as it is recorded from the pulse oximeter. FIG. 5 shows data with the amplitude normalized based on average deviation from the mean. FIG. 6 shows spliced waves laid on top of one another. FIG. 7 shows characteristic wave (+/−1 SD). FIG. 8 provides the characteristic waves from subject 1. Clearly, there is a change in waveform shape as pressure increases. In FIG. 9 I have taken the characteristic waves at a given pressure from all 10 subjects and averaged them to create characteristic waves at each pressure for the entire cohort. You can see that there had been an effect of pressure on the waveform amongst all subjects. FIG. 10 is similar to the one above, but has been created using type 1 normalization.

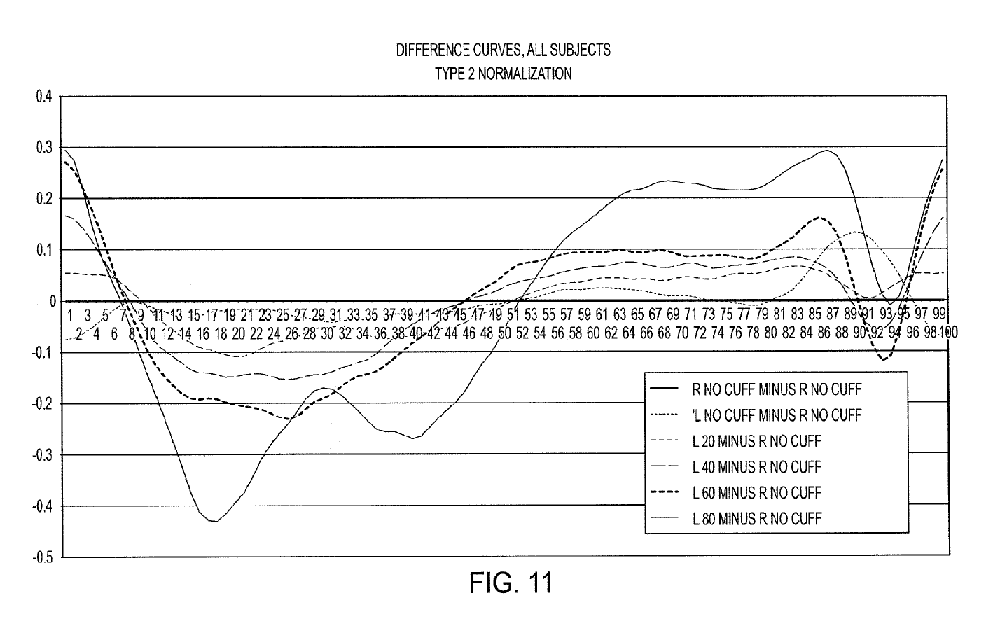

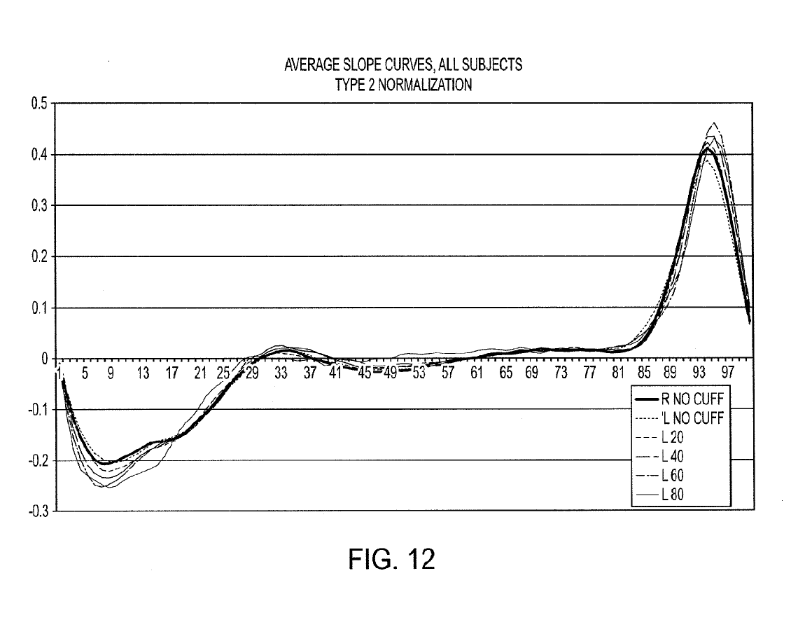

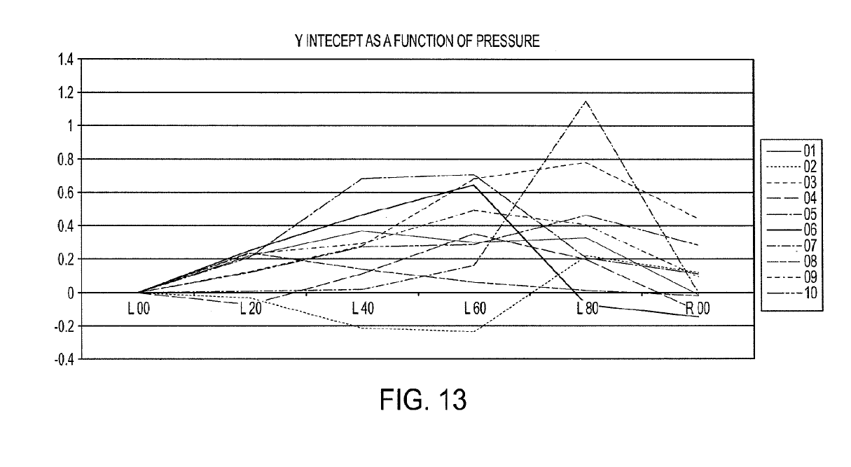

In FIG. 11 I subtracted the “R No cuff” characteristic curve from each of the other characteristic curves in order to examine the utility of using the contralateral extremity as a control. Curve subtraction requires normalization of the curves. This graph shows that there is significant potential for the contralateral extremity to serve as a control. FIG. 12 also uses the composite normalized curves from all 10 subjects at each pressure level. I have graphed the curve slope as a function of time. One of the clearest relationships revealed here is the effect of pressure on the max downslope at the beginning of the curve. The left and right signals also tend to have similar peak downslopes. In FIG. 13, I have graphed one of the type 2 normalization curve features that appears to change as a function of pressure.

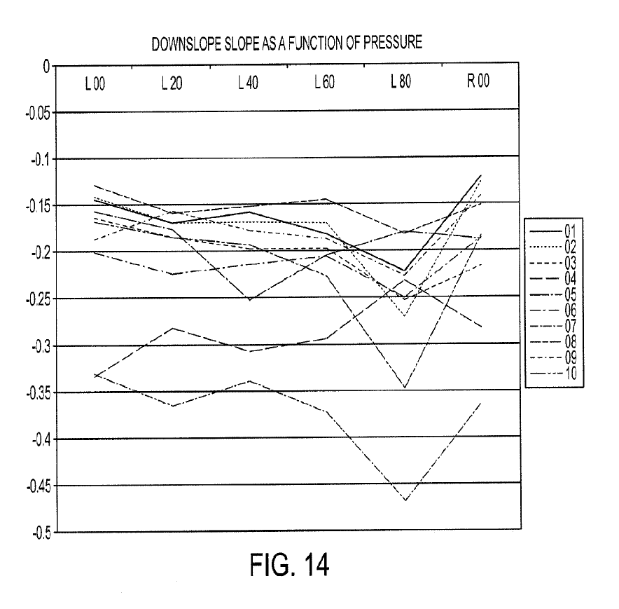

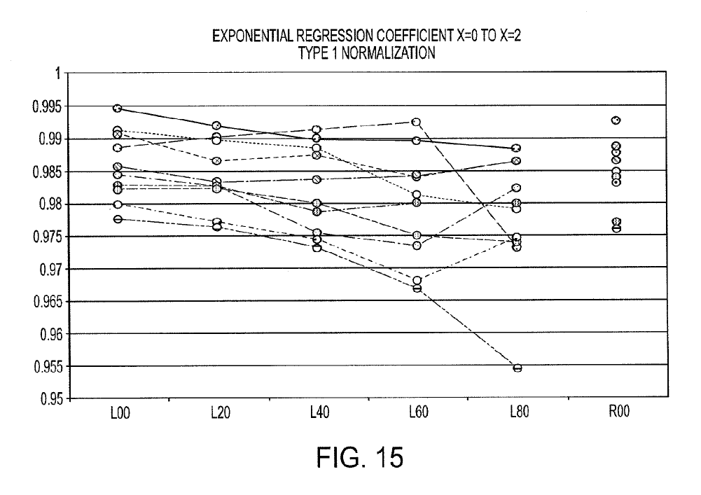

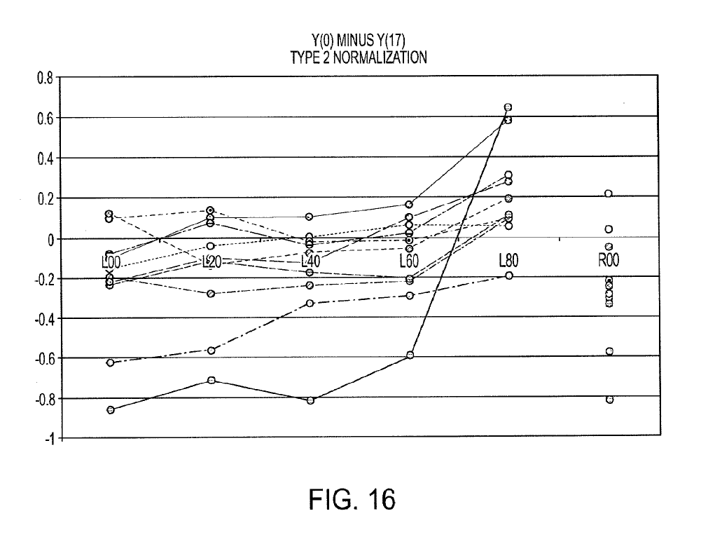

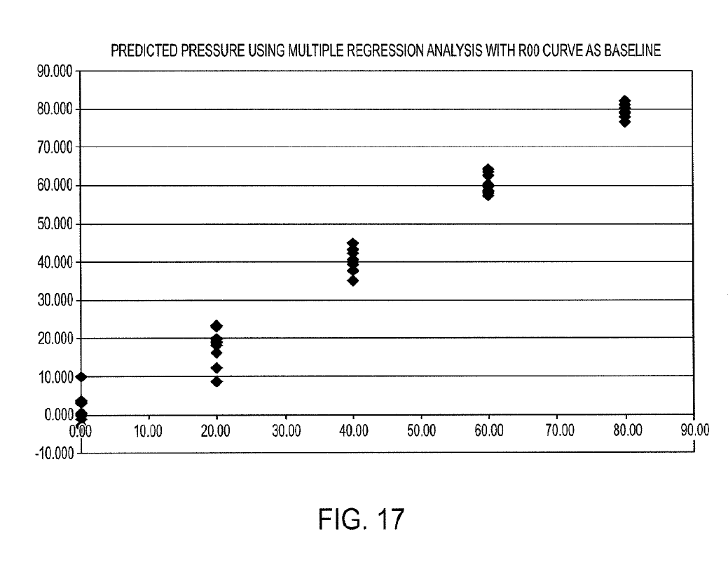

The graph in FIG. 14 shows a more detailed examination of the slope of the normalized waves around the area where their slope is the most negative. As pressure increases, this slope decreases. I fit an exponential regression to the first three data points of the normalized characteristic curves at each pressure level for each subject. I then plotted the regression coefficient as a function of pressure (FIG. 15). Again, there seems to be a relationship here. Again using the normalized characteristic curves at each pressure level for each subject, I examined the Ay between x=0 and x=17. I plotted this difference as a function of pressure (FIG. 16). In FIG. 17 I looked at almost 50 normalized curve features and then performed a multiple linear regression analysis with a backward stepwise independent variable entry method. In order to evaluate the utility of using the right-sided data as a baseline, I subtracted the R “No cuff” feature value from each of the left-sided feature values. The regression was able to reliably predict the experimental pressure.

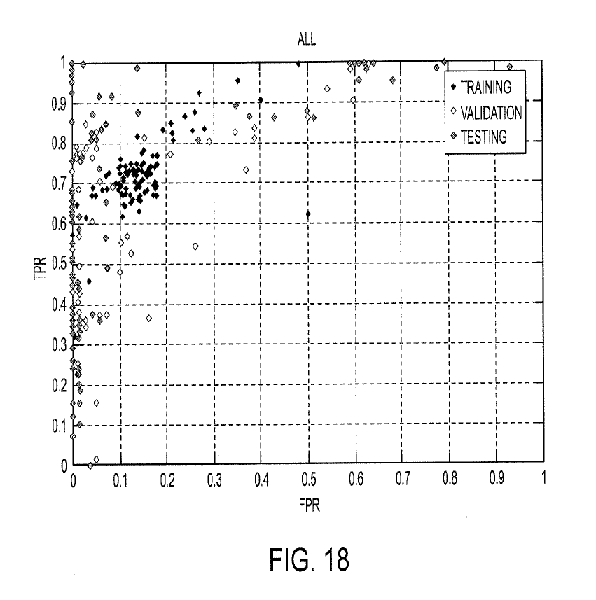

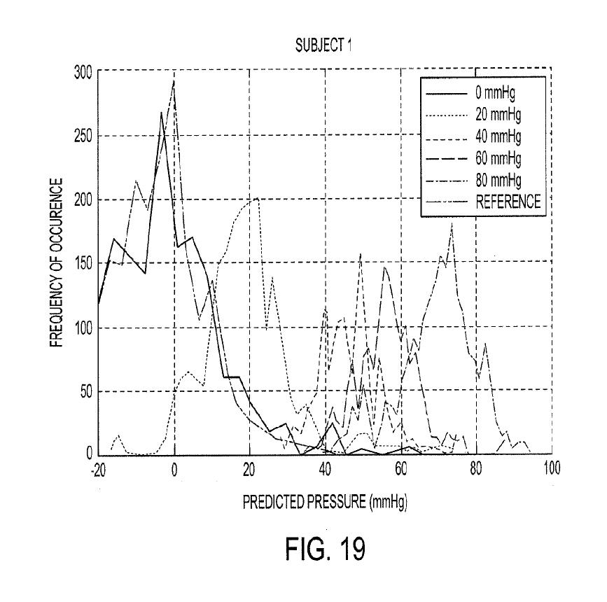

The following two graphs used a neural network that would predict the pressure for a given waveform (FIGS. 18 and 19). For each of the ten subjects, he trained the classifier on eight of the other subjects, validated the classifier on the ninth subject, and tested it on the tenth subject. He then plotted the data below to create an ROC curve (FIG. 18). We are working to improve the classifier so this data will likely improve.

He also created histograms based on the classifier output for each subject. Below is the histogram for subject 1 (one of the data sets for which the classifier performed well). Considering the classifier is predicting the pressure for a subject having “learned” on the data from the 9 other subjects, these are good results. One of the criticisms of neural networking is that it requires a lot of learning. I think these histograms will improve once we are able to capture data from more subjects (to provide more “learning” opportunity) and once we have honed the classifier parameters.

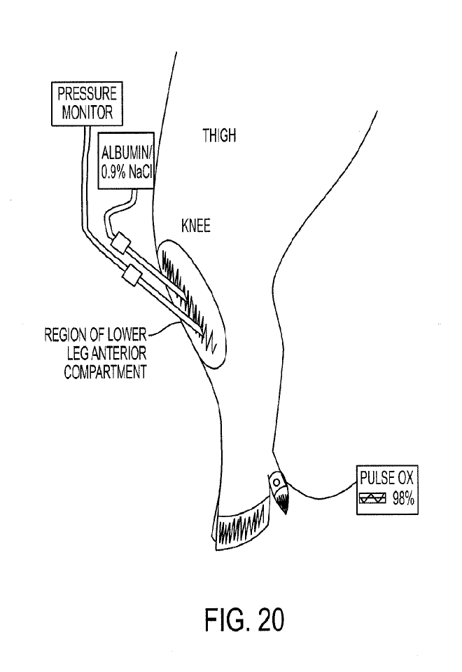

We have also collected data on two pigs, using an infusion model of compartment syndrome. This model is significantly better than our BP cuff model in terms of similarity to physiologic causes of increased compartment pressure. We captured data simultaneously from both hind limbs on an anesthetized pig (FIG. 20). We then elevated the compartment pressure in anterior compartment of one of the limbs by infusing a bovine albumin solution. By controlling the rate of albumin solution infusion we were able to titrate the compartment pressure. We were monitoring the compartment pressure continuously using the STRYKER COMPARTMENT PRESSURE MONITOR. This model has been described in the literature several times.

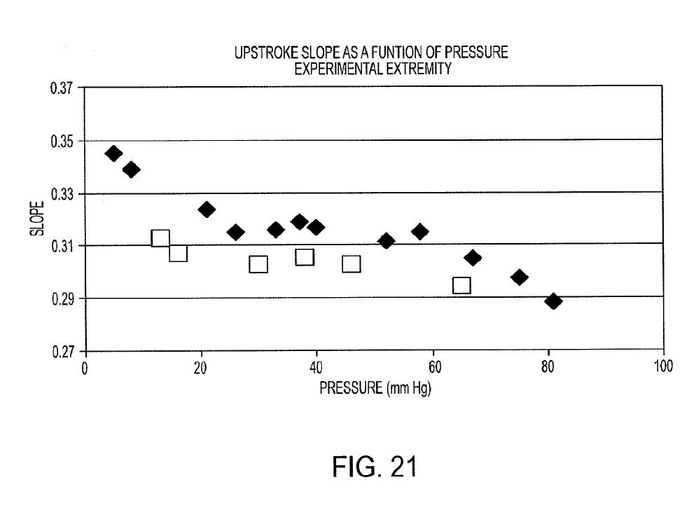

FIG. 21-23 provide some graphs I made based on data collected during our second pig pilot study. There were two phases of data collection. First we slowly increased the compartment pressure (initially by controlling the rate of infusion, then by adjusting the height of the albumin solution) then we oscillated the pressure more quickly. The blue data points represent the initial phase of data collection and the pink points represent the higher frequency pressure oscillation.

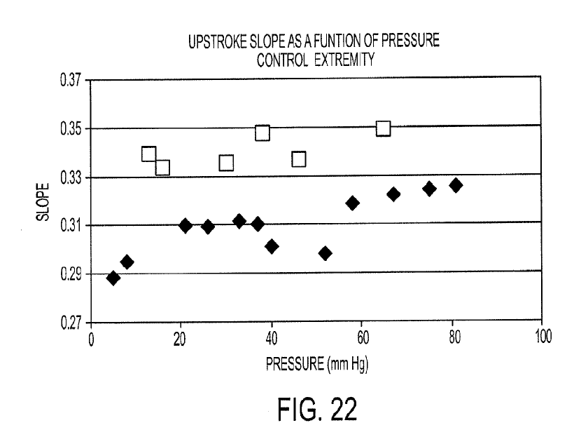

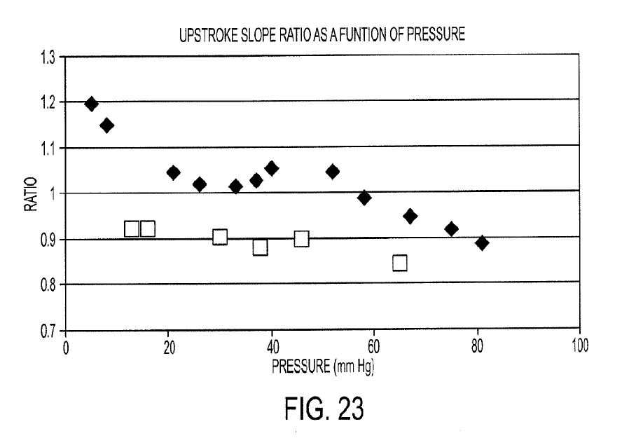

The graphs plot the upstroke slope (normalized curves) as a function of experimental limb pressure. FIG. 21 shows that the experimental limb upstroke slope decreases as experimental limb pressure increases. I speculate that the pink points show a lessened effect because there was less time for tissue equilibration during our more rapid oscillation of the pressures. FIG. 22 shows the relationship between upstroke slope (normalized curves) of the control extremity as a function of experimental extremity compartment pressure. In theory, we wouldn't expect the control extremity waveform to change as a function of contralateral extremity pressure. There was, however, a relationship here. I′m not sure if this is an artifact of the duration of the anesthesia or a demonstration of a systemic reaction to the elevated compartment pressure. FIG. 23 shows the ratio of the experimental extremity upstroke slope (normalized curves) to the control extremity upstroke slope (normalized curves) as a function of experimental extremity pressure.

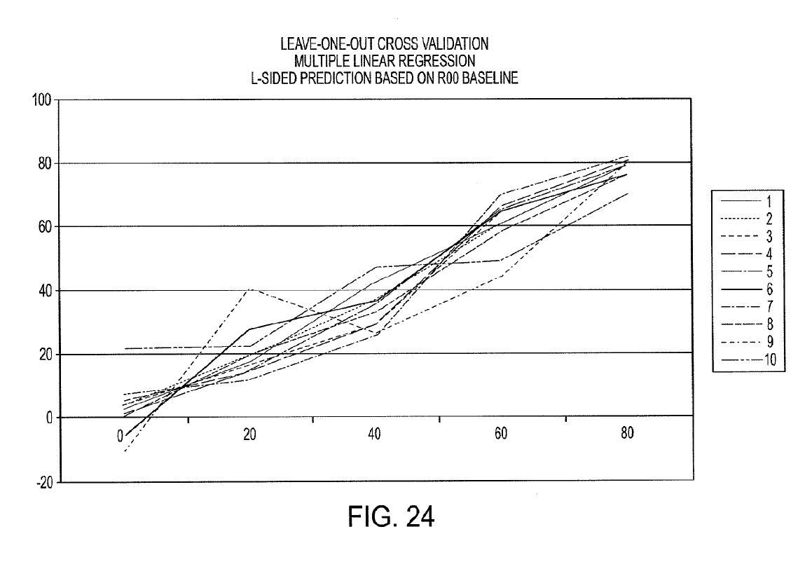

FIG. 24 shows results of a leave-one-out cross validation of the multiple regression analysis corresponding to FIG. 17.

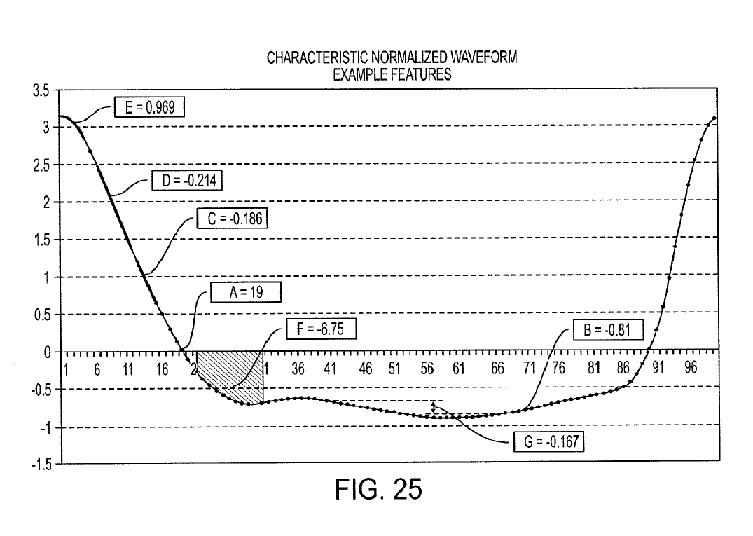

The following table summarizes waveform parameters that can be used according to some embodiments of the current invention. FIG. 25 shows an example of the various waveform parameters.

Selection of Normalized Waveform Features (not complete)

| X values | Y Values | | | | | | |
|---|---|---|---|---|---|---|---|
| at | at | Slopes at | Linear | Exponential | Area under | ΔY over | |
| specific | Specific | specific | Regression | Regression | the portions | portions of | Other Curve |
| Y values | X values | points | Coefficients | Coefficients | of the curve | the curve | paramenters |
| At initial | at x = 18 | at initial | x = 6 to x = 15 | x = 0 to x = 2 | f(60) . . . f(82) | f(1)-f(17) | range |
| y = 0 | | y = 1 | | | | | |
| (A) | | (C) | | | | | |
| at initial | at x = 70 | at initial | x = 6 to x = 11 | x = 0 to x = 4 | f(64) . . . f(78) | f(67)-f(41) | y intercept |
| y = 1 | (B) | y = 2 | (D) | | | (G) | |
| at initial | | at initial | y = 2 to y = 1 | x = 0 to x = 3 | f(68) . . . f(74) | f(67)-f(18) | R² for all or |
| y = 2 | | x = 6 | | (E) | | | portions of |
| | | | | | | | the curve |
| at second | | at initial | | x = 0 to x = 2 | f(11) . . . f(41) | f(67)-f(30) | Average |
| y = 1 | | x = 15 | | | | | deviation of |
| | | | | | | | the whole |
| | | | | | | | curve |
| at second | | at | | x = 1 to x = 4 | f(16) . . . f(36) | | average |
| y = 2 | | second | | | | | square |
| | | y = 1 | | | | | deviation of |
| | | | | | | | the whole |
| | | | | | | | curve |
| | | at | | x = 2 to x = 5 | f(21) . . . f(31) | | curve width at |
| | | second | | | (F) | | various y |
| | | y = 2 | | | | | values |
| | | | | x = 0 to y = 0 | f(0) . . . f(8) | | Area under |
| | | | | | | | the entire |
| | | | | | | | curve |
| | | | | x = 97 to x = 99 | f(95) . . . f(99) | | Upstroke |
| | | | | | | | duration |
| | | | | x = 96 to x = 99 | | | Downstroke |
| | | | | | | | duration |
| | | | | x = 95 to x = 99 | | | |
| | | | | x = 95 to x = 98 | | | |

Although the embodiments described above are in reference to monitoring and detecting elevated compartment pressure, general concepts of the current invention have additional applications. For example, embodiments of the current invention can be useful for (1) monitoring patency of revascularizations following traumatic amputation; (2) monitoring the blood supply to hands/feet/fingers/toes following surgery where blood supply may have been compromised (vascular bypass surgery, certain hand surgeries such as pollicization, syndactyly reconstruction, etc.); (3) monitoring the blood supply to rotational flaps and free flaps (often performed by plastic surgeons); (4) monitoring the blood supply to portions of the bowel following anastamosis or other surgery where blood supply is tenuous (this could be done via colonoscopy or other transrectal modality); and (5) monitoring for increased tissue pressure following cast application. Casts applied after musculoskeletal injury or after a surgical procedure can lead to increased tissue pressure due to swelling which occurs in the days following injury or surgery. This is due to the fixed volume inside the cast. Measures can be taken to allow for swelling such as “bivalving the cast”, but this compromises the structural integrity of the cast.

Concepts of the current invention include monitoring for increased tissue pressure or changes in perfusion based on distal PPG waveform amplitude, either as an absolute value or a function of time, in comparison with contra-lateral waveform amplitude, or in comparison with a reference value.

The embodiments illustrated and discussed in this specification are intended only to teach those skilled in the art how to make and use the invention and are not intended to define the scope of the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A device for identifying changes in or elevated levels of compartment pressure, comprising:
a blood-volume sensor adapted to be arranged at a distal blood-flow region relative to a region of interest; and
a data processor in communication with said blood-volume sensor,
wherein said data processor is configured to:
receive a data signal from said blood-volume sensor,
obtain a reference signal,
compare said data signal to said reference signal to identify a difference between said reference signal and said data signal that is indicative of an change in pressure within said region of interest, and
identify at least one of changes in or elevated levels of compartment pressure in said region of interest based on said comparing.

2. A device for identifying changes in or elevated levels of compartment pressure according to claim 1, further comprising a data storage unit configured to store said reference signal, wherein said data processor is configured to obtain said reference signal from said data storage unit.

3. A device for identifying changes in or elevated levels of compartment pressure according to claim 2, wherein said stored reference signal comprises previously obtained baseline data.

4. A device for identifying changes in or elevated levels of compartment pressure according to claim 1, wherein said data processor is configured to generate a reference signal based on a model.

5. A device for identifying changes in or elevated levels of compartment pressure according to claim 1, further comprising a second blood-volume sensor adapted to be arranged at a reference region to provide said reference signal.

6. A device for identifying changes in or elevated levels of compartment pressure according to claim 1, wherein said blood-volume sensor takes the form of at least one selected from a group of a non-invasive blood-volume sensor and an optical blood-volume sensor.

7. (canceled)

8. A device for identifying changes in or elevated levels of compartment pressure according to claim 5, wherein said second blood-volume sensor takes the form of at least one selected from a group of a non-invasive blood-volume sensor and an optical blood-volume sensor.

9. (canceled)

10. A device for identifying changes in or elevated levels of compartment pressure according to claim 1, wherein said data processor is configured to calculate a first ratio of amplitudes of said data signal and said reference signal at a first time and calculate a second ratio of amplitudes of said data signal and said reference signal at a second time to determine a change of said second ratio relative to said first ratio.

11. A method of detecting and monitoring changes in or elevated levels of compartment pressure, comprising:
arranging a blood-volume sensor at a distal blood-flow region relative to a region of interest, said blood-volume sensor providing a data signal;
obtaining a reference signal;
comparing said data signal to said reference signal to identify a difference between said reference signal and said data signal that is indicative of a change in pressure within said region of interest; and
identifying at least one of changes in or elevated levels of compartment pressure in said region of interest based on said comparing.

12. A method of detecting and monitoring changes in or elevated levels of compartment pressure according to claim 11, wherein said obtaining said reference signal comprises at least one of retrieving said reference signal from a data storage unit, generating said reference signal based on a model or obtaining said reference signal as a second data signal from a reference region.

13. A method of detecting and monitoring changes in or elevated levels of compartment pressure according to claim 12, wherein said reference signal comprises previously obtained baseline data.

14. A method of detecting and monitoring changes in or elevated levels of compartment pressure according to claim 11, further comprising arranging a second blood-volume sensor at a reference region to provide said reference signal.

15. A method of detecting and monitoring changes in or elevated levels of compartment pressure according to claim 11, wherein said blood-volume sensor takes the form of at least one selected from a group of a non-invasive blood-volume sensor and an optical blood-volume sensor.

16. (canceled)

17. A method of detecting and monitoring changes in or elevated levels of compartment pressure according to claim 14, wherein said second blood-volume sensor takes the form of at least one selected from a group of a non-invasive blood-volume sensor and an optical blood-volume sensor.

18. (canceled)

19. A method of detecting and monitoring changes in or elevated levels of compartment pressure according to claim 11, wherein said comparing said data signal to said reference signal comprises calculating a first ratio of amplitudes of said data signal and said reference signal at a first time and calculating a second ratio of amplitudes of said data signal and said reference signal at a second time to determine a change of said second ratio relative to said first ratio.

20. A computer-readable medium comprising software, when executed by a computer, said software causes the computer to perform processes comprising:
receiving a data signal from a blood-volume sensor arranged at a distal blood-flow region relative to a region of interest;
obtaining a reference signal;
comparing said data signal to said reference signal to identify a difference between said reference signal and said data signal that is indicative of a change in pressure within said region of interest; and
identify at least one of changes in or elevated levels of compartment pressure in said region of interest based on said comparing.

21. A computer-readable medium according to claim 20, wherein said obtaining said reference signal comprises at least one of retrieving said reference signal from a data storage unit, generating said reference signal based on a model or obtaining said reference signal as a second data signal from a reference region.

22. A computer-readable medium according to claim 21, wherein said reference signal comprises previously obtained baseline data.

23. A computer-readable medium according to claim 20, further comprising receiving a second data signal from a second blood-volume sensor arranged at a reference region to provide said reference signal.

24. A computer-readable medium according to claim 20, wherein said blood-volume sensor takes the form of at least one selected from the group of a non-invasive blood-volume sensor and an optical blood-volume sensor.

25. (canceled)

26. A computer-readable medium according to claim 23, wherein said second blood-volume sensor takes the form of at least one selected from a group of a non-invasive blood-volume sensor and an optical blood-volume sensor.

27. (canceled)

28. A computer-readable medium according to claim 20, wherein said comparing said data signal to said reference signal comprises calculating a first ratio of amplitudes of said data signal and said reference signal at a first time and calculating a second ratio of amplitudes of said data signal and said reference signal at a second time to determine a change of said second ratio relative to said first ratio.

29. A device for identifying hemodynamic changes, comprising:
a blood-volume sensor adapted to be arranged at a distal blood-flow region relative to a region of interest; and
a data processor in communication with said blood-volume sensor,
wherein said data processor is configured to:
receive a data signal from said blood-volume sensor,
obtain a reference signal,
compare said data signal to said reference signal to identify a difference between said reference signal and said data signal that is indicative of an hemodynamic change within said region of interest, and
identify said hemodynamic change in said region of interest based on said comparing.
